(19) United States
(12) Patent Application Publication
(10) Pub. No.: US 2006/0019375 A1
(43) Pub. Date: Jan. 26, 2006

(54) Device for culturing cells, particularly human or animal cells

(76) Inventors: Josef Seidl, Aldersbach (DE); Wilhelm Scherze, Furth (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/528,056

(22) PCT Filed: Sep. 16, 2002
(86) PCT No.: PCT/EP02/10358

§ 371 Date: Mar. 15, 2005

(51) Int. Cl. C12M 3/00

(52) U.S. Cl. 435/289.1

(57) ABSTRACT

The invention relates to a device (30) for culturing cells of the most diverse type, particularly human or animal cells. According to the invention, a culture is prepared from cells of at least one specified type in a defined environment, and the cells of the relevant culture are supplied with assigned, liquid nutrient media, growth factors, gases and the like. Cell culturing and incubating equipment is provided that is configured for enabling, in at least one cell culture chamber (20) of the device (30), the culturing of cells that adapt themselves in an almost optimal manner to their living and growth conditions required in individual circumstances.

The invention relates to a device for cultivating cells of the most diverse type, particularly human or animal cells; one culture each is prepared from cells of at least one specific type in a defined environment and the cells of the relevant culture are supplied with assigned, liquid nutrient media, growth factors, gases or the like in the process.

In general, cultures of the above-mentioned type are prepared from individual cells that either result from parts of tissue, primary cultures, from cell lines or cell stems obtained by enzymatic, mechanic or chemical disintegration.

With previously known cell cultivation devices, culture vessels made of plastic that are incubated in CO₂ incubators are normally used for preparing the cultures. These guarantee a constant temperature (e.g. 37° C.) and a buffering of the medium by way of a 5 to 10 percent CO₂ gassing. Oxygen supply is effected by simple diffusion. With the known methods and equipment for the cultivation of cells, co-cultivation and freely changeable incubation conditions are normally not possible.

For microscopic observation or for specific examinations, the culture vessels have to be taken out of the relevant incubators; incubation is interrupted, the cells cool down and the test conditions are no longer constant as a result.

The previously known methods and devices for cultivating cells, however, do no longer meet the requirements of modern cell culture technology.

With regard to current main research areas in the pharmaceutical industry in particular, these being the fields of inflammation (rheumatics), the fight against cancer, cardiovascular diseases, Aids, apoptosis (programmed cell death) and blood coagulation, the development and the testing of suitable new active agents and drugs by means of a new device for cultivating cells that is substantially improved and enables the testing of substances and actions under almost in-vivo conditions, that means with an almost perfect mapping of complex, biological systems, before passing on to the clinical phases (tests on test persons) is indispensable.

Out of consideration for the above-mentioned situation, there is demand for a method of simulating the progress of reactions within one or several organ system(s) (e.g. by means of a series connection of cell culture chambers with hepatocytes and other types of cells, testing for degradation products and metabolites) in order to considerably minimize, on the one hand, the period of time that passes between the identification of the action of a substance and drug approval and to enable the obtaining of necessary findings on the mechanism of action of the substance within a complex biological system before passing on to the clinical test phase, on the other hand.

A similar situation is given, for example, in the area of cosmetics industry as well.

State-of-the-art technology includes, for example, multivalent cell culture systems (see DE 199 15 178 A1, for example), problem-adapted cell culture systems for specific tasks (see WO 98/17822 for example) or methods for the replication of cell cultures (see WO 97/37001 for example).

Furthermore it is known, for example, from WO 99/23206 that there is a method of mixing a cell culture infected with varicella in cylindrical bottles, for example.

Finally a method and a device used to take up a cell culture are known from EP 0 999 266 A1; these aim at creating the most homogenous possible conditions for a molecular biological or genetic examination of cells.

Out of consideration for the situation in the field of modern cell culture technology described in the beginning, the target of the present invention is now to create a new, improved method of cultivating cells of the most diverse type, particularly human or animal cells, that does away with the disadvantages of the previously known systems and devices for cell cultivation and offers, in particular, the opportunity to simulate highly complex, biological processes in real time and under almost in-vivo conditions (i.e. like in a living organism) under so-to-speak ideally adjusted living and growth conditions for the cells.

The task defined above is solved on the basis of a device for cultivating cells of the most diverse type, particularly human or animal cells, one culture each of at least cells of one specific type being prepared in a defined environment and the cells of the relevant cultures being supplied with assigned, liquid nutrient media, growth factors, gases and the like, by the invention by way of equipping the device with cell cultivation media and incubation media of a kind that enables the cells established in at least one cell culture chamber to set the conditions for living and growth that are necessary in the individual cases themselves.

The device according to the invention preferably has the combination of the following characteristics:
a) Equipment serving to start a flow of freely selectable, defined liquid media into the at least one cell culture chamber in order to continuously supply the cells that have been established there;
b) Equipment serving to start a flow of different gases with freely selectable concentrations into at least one cell culture chamber in order to effect a constant, continuous gassing of the cells that have been established there;
c) Equipment serving to effect a regulated and/or controlled heating of the at least one cell culture chamber in such a manner so as to ensure a constant temperature therein over the duration of an experiment;
d) Equipment for the permanent microscopic observation of the cells established in the at least one cell culture chamber without samples of the cell culture being removed over the duration of an experiment;
e) Equipment for a permanent measuring of all relevant cell culture parameters using corresponding sensors that are integrated inside the at least one cell culture chamber; and
f) A feedback control medium assigned to the at least one cell culture chamber, serving to optimize the incubation conditions in the at least one cell culture chamber.

The relevant cell culture parameters are, in particular, pH values, glucose, lactate, oxygen, electric potential and the like.

As far as the device according to the invention is concerned, a given number of cell culture chambers is provided preferentially that may either be connected in series or in parallel; a corresponding number of cell cultures is preferably established inside this given number of cell culture chambers at the same time.

The device for cultivating cells according to the invention ensures in particular that the cells of all cultures are continuously supplied with liquid nutrient media, growth factors, gases or the like, without the cells of a culture having to be taken out of their habitual, defined environment, while all cell cultures can be permanently observed by microscope without an interruption of gassing at the same time.

According to a further embodiment of the device according to the invention, equipment that serves to vary the type of liquid media and/or their directions of flow and/or their distribution and/or their flow volumes for the duration of an experiment are provided. In addition, however, equipment that serves to vary the type of gases and/or their directions of flow and/or their distribution and/or the gassing concentrations for the duration of an experiment can also be provided.

The above-mentioned variation options guarantee an extremely flexible mode of operation of the device according to the invention.

When the cell culture chambers of the device according to the invention are connected in series, the liquid media and/or the gases, for example, can be passed on continuously from cell culture chamber to cell culture chamber.

In order to ensure constant temperatures in the individual cell culture chambers for the device for cultivating cells according to the invention over the duration of an experiment, the device is preferably equipped with equipment that serves to permanently measure the temperatures inside the individual cell cultures and to enter them into a corresponding temperature adjustment circuit and/or temperature control circuit as actual temperature values so that the heating of the relevant cell culture chamber can be adjusted and/or controlled accordingly.

As will be explained in more detail below, each cell culture chambers is fitted with its own heating for this purpose, while one infrared temperature measuring device each is fitted above the relevant cell culture chamber; this device measures the temperature in the relevant cell culture and reports this measured temperature value to a monitoring and control system. If the temperature that was preset at the beginning changes in the at least one cell culture chamber, the temperature adjustment and/or control circuit reduces or increases the heating power of the heating of the relevant cell culture chamber.

The temperatures can be measured by means of other temperature sensors as well, however.

As will be explained in more detail below as well, the temperatures in the individual cell culture chambers can be freely adjusted and changed by means of the monitoring and control system over the entire duration of an experiment for reasons of flexibility.

A further, particularly advantageous embodiment of the method provides that it has at least one cell culture chamber in which a gas-permeable membrane is arranged in such a way that one cell culture each of a different type can be established on both sides of this membrane for the purpose of a direct co-cultivation of both cell cultures; equipment for starting a first flow of media to the first side of the membrane, i.e. the apical side with the first cell culture, and a second flow of media that differs from the first flow of media to the other side of the membrane, i.e. the basolateral side with the second cell culture, are provided.

In this way, the cells growing on the apical side act as a covering layer, while the cells on the basolateral side act as interior cells. The cells of the first cell culture and the cells of the second cell culture are in a relatively close contact to each other via the membrane so that it is possible to examine exchange processes inside the layers on the apical side and on the basolateral side.

Furthermore it is also possible to examine a potential exchange of effective, bio-active molecules (e.g. growth factors, hormones, etc.) in the course of such a co-cultivation if gas-permeable membranes with different, selectable pore sizes are used for the device according to the invention. These kinds of examinations are particularly important for parts of tissue consisting of different types of cells, such as the transition endothelium cells-fibroblasts (blood vessels) or mucous membrane cells-fibroblasts (intestine, stomach) for example.

Besides the device according to the invention can be used particularly advantageously for an indirect co-cultivation by means of connecting different biological systems (i.e. types of tissue/cells) in series in suitable cell culture chambers.

In that way, it is possible to reproduce, so to say, complete organ systems and to examine the relevant metabolic processes. These measures can be explained in more detail using an example: A substance that is normally not toxic is absorbed via the digestive tract and gets into the liver via the bloodstream. The liver cells catabolize the substance into catabolic products that may have a toxic effect under certain circumstances. In order to check this, the “suspect” substance is placed in an incubation chamber that is populated with hepatocytes (liver cells). Via a defined nutrient media supply (media flow=“blood vessel”), possibly toxic catabolic products get into a cell culture chamber that is connected to the other chamber; when nerve cells, for example, die off there, it can be inferred that the substance is neurotoxic.

According to a further, extremely advantageous embodiment of the device according to the invention, a video-supported microscopic observation system for observing the at least one cell culture in the at least one cell culture chamber is provided; this will also be described in more detail below.

Finally there is another advantageous embodiment of the device according the invention that includes a computer-controlled monitoring and control system; all data that are obtained by means of
A permanent microscopic observation of the at least one cell culture inside the at least one cell culture chamber and/or
A permanent measuring of the relevant cell culture parameters and/or
A permanent measuring of the temperature in the at least one cell culture inside the at least one cell culture chamber,
can be transferred there for further processing and for a corresponding actuation of the feedback control elements.

The feedback control elements are in particular control algorithms that are included in a data processing system of the computer-controlled monitoring and control system.

In this connection, a software-assisted measuring system is provided for a permanent measuring of the relevant cell culture parameters.

A continuous measuring of cell culture parameters can be carried out preferably by means of specific probes and/or sensors; for pH value, lactate, electric potential and the like, for example; these measurements can be evaluated and represented by a corresponding software. This type of measurement delivers more exact results compared to conventional methods, and this enables you to analyze certain questions that you have not been able to analyze with measuring methods used up to now.

By means of a software-aided measuring that is used for the device according to the invention, for example, certain tests on animals in the pre-clinic phase can be replaced for the most part.

In summary, the device for cultivating cells according to the invention offers the following benefits in particular:
1. Possibility of connecting in parallel a given number of cell culture chambers inside the device for reference measurements;
2. Possibility of connecting in series a given number of cell culture chambers inside the device for organ simulation.
3. Possibility of a variable temperature adjustment and/or control.
4. Possibility of a variable gassing of the individual cell culture chambers.
5. Possibility of an individual supplying of the cell cultures with nutrient substances and/or active agents.
6. Possibility of a permanent microscopic observation inside the individual cell culture chambers and a corresponding video recording without an interruption of the cell cultivation process.
7. Possibility of a permanent measuring of various cell culture parameters by means of an integrated sensor system.
8. Making available of a high-quality reusable system, that means processing of stainless steel and quartz glass that are of a fully autoclavable structure in order to reduce waste.

In the following, the invention is explained in more detail on the basis of examples of application:

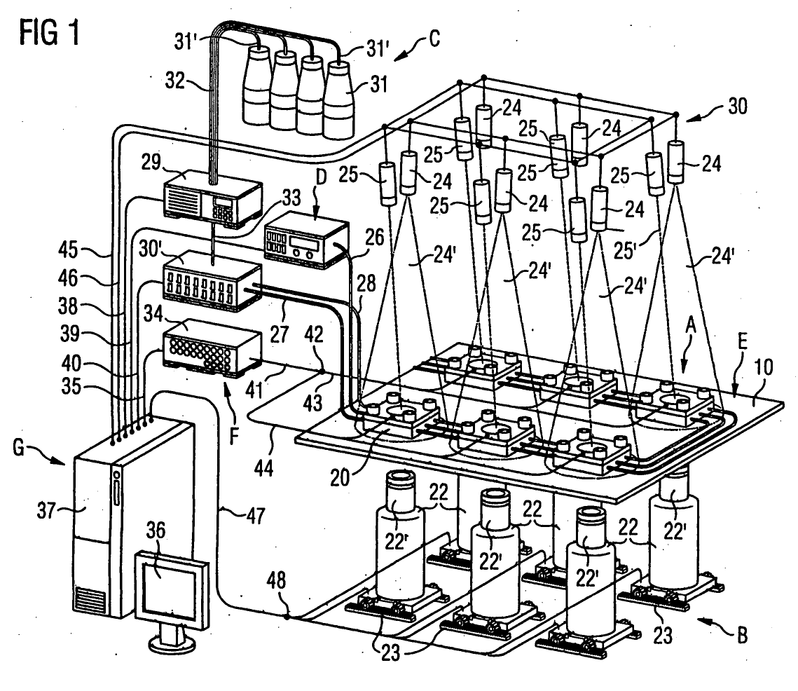

FIG. 1 showing a diagram of a device for cultivating cells of the most diverse type, particularly human or animal cells; and

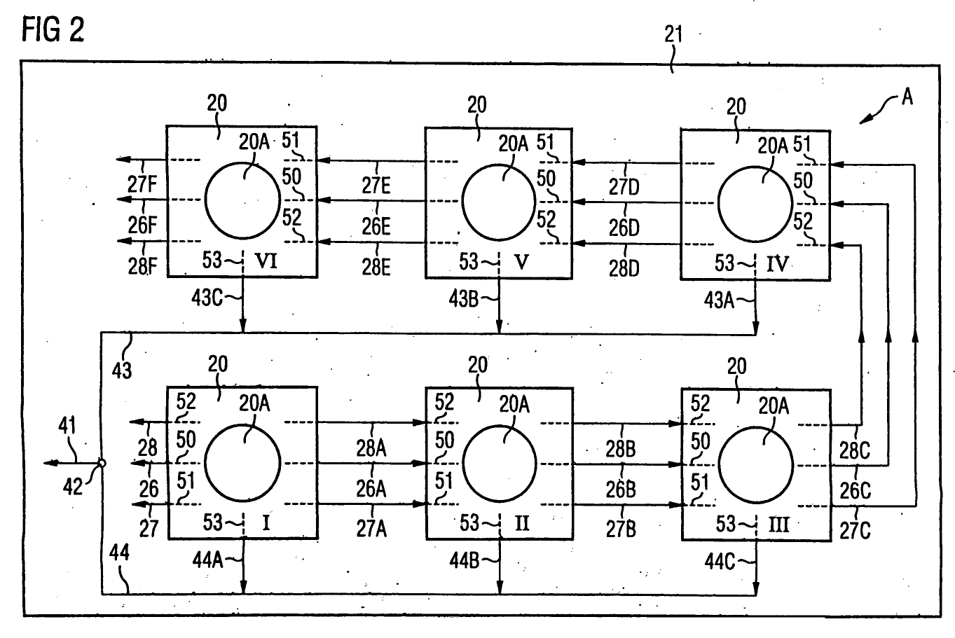

FIG. 2 showing a diagram of a cell culture chamber group arranged on the basis of the device according to FIG. 1; this group consists of a given number of individual cell culture chambers.

FIG. 1 shows a schematic view of the device 30 for cultivating cells of the most diverse type; one culture each of cells of at least one specific type being prepared in a defined environment inside an assigned cell culture chamber and the cells of the relevant culture being supplied with pre-selected, liquid nutrient media, growth factors, gases and the like.

Altogether, the device 30 is designed in such a way that it is equipped with cell cultivation and incubation equipment of such a quality that it enables the cells established in the cell culture chambers of the device 30 to set the living and growth conditions that are necessary in the individual case themselves, this means, in particular, that this aims, so to speak, at an optimization of these living and growth conditions.

In the device 30 presented in FIG. 1, for example, six cell culture chambers 20 are positioned in the form of a cell culture chamber group A on an accordingly assigned base 21. In particular, the base 21 constitutes a heating system E for incubation that ensures constant temperatures inside each of the cell culture chambers 20 during the operating period of the device 30.

This heating system E is preferably used for the electrical heating of the relevant cell culture chamber 20, and it enables a very exact temperature control. This heating system E is, in particular, designed in such a way that each individual cell culture chamber 20 of the cell culture chamber group A is equipped with its own heating that is integrated in the base 21.

It is a particular advantage of the heating system E that it can be controlled by means of an assigned software. For this purpose, a system of infrared temperature measuring devices 25 is fitted above the cell culture chamber group, each individual cell culture chamber 20 being assigned to a corresponding infrared temperature measuring device 25. The relevant infrared temperature measuring device 25 reads the temperature prevailing in the cell culture in the relevant cell culture chamber 20 and permanently reports the relevant measuring result to a computer-controlled monitoring and control system G that mainly consists of a data processing system 37 and an accessory monitor 36. The individual infrared temperature measuring devices 25 are connected to the monitoring and control system G via a joint connecting line 45. When the initially pre-set temperatures in the cell culture chambers 20 of the cell culture chamber group A change, the heating system E is controlled and/or adjusted automatically via the monitoring and control system G, this means that the temperature in the individual cell culture chamber 20 is permanently adjusted to a constant temperature.

It would also be possible to measure the temperature in the individual cell culture chambers 20 by means of other temperature sensors instead of infrared temperature measuring devices.

Furthermore the software contained in the monitoring and control system can be used to ensure that the temperatures in the individual cell culture chambers 20 of the cell culture chamber group A can be freely set and selected over the duration of the entire experiment if this becomes necessary for specific reasons.

For the purpose of a permanent, video-supported, microscopic observation of the inside of the relevant cell culture chamber 20, a video system B with an accordingly assigned microscope system is provided. This video system B will be explained in more detail in the following.

Below every individual cell culture chamber 20 of the cell culture chamber group A that includes a total of six cell culture chambers in this application example, a video camera 22 with a microscope adapter 22′ is fitted on a mechanically adjustable, mobile table, therefore there is a total of six video cameras 22 with accessory microscope adapters 22′. Thus one video camera 22 each with a microscope adapter 22′ serves to observe one cell culture chamber 20 each. After the experiment has been started and after meaningful areas in the cell culture contained in the relevant cell culture chamber 20 have been identified, an observation sector in the cell culture chamber 20 is determined. The mechanically adjustable mobile table 23 is moved to this observation sector then by means of adjusting screws (not represented), then the mobile table 23 is locked and the video system B remains in the same position over the entire duration of the experiment as a result. Furthermore the definition setting at the relevant microscope adapter 22′ is adjusted at the start of the test. This adjustment process on the relevant microscope adapter 22′ is carried out for all six cell culture chambers 20 and then remains unchanged until the experiment has been completed.

The video system B is preferably controlled via the software contained in the monitoring and control system G as well. Every individual video camera 22 with microscope adapter 22′ is controlled in the process. This is carried out in particular in such a way that pictures of the relevant cell culture in the cell culture chamber 20 are taken at freely selectable intervals (every minute, for example, a light source 24 fitted above the relevant cell culture chamber 20 illuminating the relevant cell culture at the relevant point in time at which such a recording is made, so that a sufficient illumination of the inside of the cell culture chamber 20 is ensured for the video recordings. When the video recording is completed, the control switches off the relevant light source 24 until the next video recording is made. The light beam and/or light cone that is emitted by each of the light sources 24 and that enters inside the relevant cell culture chamber 20 through a suitable pane of glass (not represented) is marked 24′ in FIG. 1.

All light sources 24 are connected to the monitoring and control system G via a joint connecting line 46.

Every single light beam/light cone 24 illuminates the entire area of the cell culture contained in the relevant cell culture chamber 20. This is a method of transillumination.

Instead of using such a method of transillumination, however, the light sources used to illuminate the cell culture contained in the relevant cell culture chamber 20 might also be fitted directly on the relevant assigned video camera 22 and/or the relevant assigned microscope adapter 22′, the transillumation method being replaced by a top view method in this way.

The video system B is also connected to the monitoring and control system G via a line 47; from the monitoring system, the line 47 is connected to a junction point 48 to which the individual video cameras 22 are connected via correspondingly assigned lines.

The video system B with microscope system as described above is only one the possible models. Another possible embodiment of such a system for the permanent observation of the inside of the cell culture chambers comprises a single observation system, consisting of a video camera and a microscope adapter, installed on a mobile table; this mobile table moves to the six cell culture chambers 20 of the cell culture chamber group A at freely selectable intervals. The adjustment of the observation system is carried out for the individual cell culture at the start of the test, this means preferably after meaningful areas have been identified in the relevant cell culture, by means of the respective software included in the monitoring and control system G, this means that the six target positions of the moving table on which the observation system has been mounted are programmed by means of the respective software. On account of the mechanic tolerances of the moving table, however, it is necessary to include an area that is larger than the area inside the individual cell culture chamber 20 to be observed. The software now serves to define the area to be observed within this larger area. The software is able to record and to recognize contours, this means that the contours and the configuration of the cells is recognized when the table moves in the direction of a cell culture chamber again and an initially defined observation area is recorded.

This observation system that has been explained last is not represented in detail in the drawings, but the individual cell culture chambers 20 are also illuminated by means of the light sources 24, as it has already been explained in detail above.

In this case, it is also possible to replace the transillumination method by a top view method, this means that the light sources used to illuminate the cells contained in the individual cell culture chamber 20 can be mounted directly on the relevant assigned video camera 22 and/or the relevant assigned microscope adapter 22′.

Furthermore the device 30 represented in FIG. 1 is equipped with a dosage system C for liquids (e.g. liquid nutrient media and the like) that is fitted with e.g. four liquid storage tanks 31 with one assigned liquid take-off line 31′ each; these liquid take-off lines 31′ constitute a group of lines 32. This group of lines 32 is, on the other hand, connected to a pump system 29 through which the different cell culture chambers 20 of the cell culture chamber group A are supplied with freely selectable liquids that are contained in the liquid storage tanks 31.

The pump system 29, on the other hand, is connected to a multi-valve module 30′ via a line 33. The liquids are supplied to the cell culture chamber group A from the multi-valve module 30′ via sterile hose line systems 27 and 28; these liquids are passed on from the individual cell culture chambers 20 in a flexible manner, this means from one cell culture chamber to the next, as will be explained in more detail by means of FIG. 2 below.

The liquid supply as well as the passing on of liquids is carried out via sterile hose systems that are installed with standard hose connecting elements and distributors at the start of a test; this means that they are connected to corresponding supply channels of a relevant cell culture chamber 20. Here the connection of the standard hose elements (not represented in detail in the drawings) with the assigned supply channels of the relevant cell culture chamber 20 is adjusted in such a way that sterility is guaranteed.

For reasons of flexibility, the types of liquids and/or their directions of flow and/or the distribution of liquids and/or their flow volumes can be changed and/or controlled during a test; this is preferably controlled by the computer-controlled monitoring and control system G. For this purpose, the pump system 29 is connected to the monitoring and control system G via a connecting line 38 and the multi-valve module 30′ via a connecting line 40.

Therefore the dosage system C of the cell culture system 30 enables you to supply the cell culture chamber group A with a variety of different liquids.

Furthermore the device 30 is equipped with a gassing system D for a variety of different gases. This gassing system D serves to gas the different cell culture chambers 20 of the cell culture chamber group A with a variety of different gases, e.g. air, O₂, N₂, CO₂. From the gassing system D, the gas is supplied to the cell culture chamber group A via a sterile hose line 26. Here the gases can be passed on from the different cell culture chambers 20 in a flexible manner as well, by means of accordingly assigned supply channels, this means from one cell culture chamber to the next (see FIG. 2).

The gas is altogether supplied and passed on via sterile tubes that are installed by means of standard hose connecting elements and distributors at the start of a test. The connections of the hose connecting elements with the correspondingly assigned supply channels of a relevant cell culture chamber 20 are adjusted in such a way so that sterility is guaranteed. With the gassing system D as well, the types of gases and/or the directions of flow and/or the gas distribution and/or the gassing concentration can be changed and/or controlled during an experiment for reasons of flexibility. For this purpose, the gassing system D, on the other hand, is connected to the monitoring and control system G, that contains the relevant software for controlling the gassing system D, via a connecting line 39.

Finally the device 30 for cultivating cells further includes a monitoring system F with predefined sensor modules 34. By means of this monitoring system F, the relevant parameters in the relevant cell culture chamber 20 of the cell culture chamber group A can be measured, measured permanently in particular, using accordingly assigned sensors, for the entire duration of a test; these parameters being, for example, pH value, glucose, lactate, oxygen, electric potential, etc. For this purpose, the monitoring system F is connected to the individual cell culture chambers 20 of the cell culture chamber group A of the cell culture system 30 via a line 41, via a junction point 42 and from there via further lines 43 und 44 and accordingly assigned branch lines.

The parameters measured by the sensors (not shown) are transmitted by the monitoring system F via a line 35 to the computer-controlled monitoring and control system G for further processing and for a subsequent actuation of the feedback control elements.

Each cell culture chamber 20 is equipped with corresponding sensor connecting channels; this will be explained in detail below. The sensors and the relevant assigned channels are adjusted to each other in such a way so as to guarantee sterility.

A particularly advantageous design of the monitoring system F in connection with the computer-controlled monitoring and control system G enables a permanent measuring of the relevant cell culture parameters by means of a software-aided measuring method (as already explained above).

FIG. 2 shows a schematic top view of the cell culture chamber group A of the cell culture system according to FIG. 1. With this cell culture chamber group A arranged on the base 21, a total of six cell culture chambers 20 is connected in series, so to speak, in such a way that the liquid media as well as the gases can be passed on from one cell culture chamber 20 to the next, this means it can be passed on continuously to the relevant subsequent cell culture chamber 20.

In each of the six cell culture chambers 20, at least one cell culture to be examined is established; for the sake of simplicity, however, this example of application will be based on six cell cultures the relevant cells of which have to be supplied with defined liquid nutrient media, growth factors, gases and the like.

For this purpose, a flow of freely selectable, defined, liquid media on the one hand, and a flow of different gases with freely selectable concentrations on the other hand, to the six cell culture chambers 20 of the cell culture chamber group A is started; as already explained above, the liquids are primarily supplied to the cell culture chamber group A from the multi-valve module 30′ according to FIG. 1 via the sterile tube line systems 27 and 28, while the gas is supplied to the cell culture chamber group A from the gassing system D according to FIG. 1 via the sterile hose line 26 at the same time.

For reasons of clarity, the six cell culture chambers 20 that are subsequently connected in series are marked I, II, III, IV, V and VI.

The hose line systems 27 and 28 for liquids and the hose line 26 for gases are directly connected to the first cell culture chamber 1; the result is that the hose line 26 directly discharges into a gas channel 50 inside this first cell culture chamber I, whereas the hose tube system 27 discharges into a corresponding liquid channel 51 and the hose line system 28 discharges into a liquid channel 52 inside this first cell culture chamber 1 at a time. In this way, the cell culture contained in the first cell culture chamber I is supplied with liquid media and gases, and then the subsequent cell culture chambers II to VI are correspondingly supplied with liquid media and gases successively. In detail, the cell culture chamber I is connected, via the liquid hose lines 27A and 28A and via a gas hose line 26A, to the second cell culture chamber II, that is again connected, via the liquid hose tubes 27B and 28B and a gas hose line 26B, to the third cell culture chamber III, that is again connected, via the liquid hose tubes 27C and 28C and a gas hose tube 26C, to the fourth cell culture chamber IV, that is again connected, via the liquid hose lines 27D and 28D as well as a gas hose line 26D to the fifth cell culture chamber V, that is finally connected, via the liquid hose lines 27E and 28E and via a gas hose line 26E, to the sixth cell culture chamber VI.

On account of this connection of the six cell culture chambers 20 in series, that means of chambers I to VI, each liquid hose line 28A or 28B or 28C or 28D or 28E respectively discharges into one liquid channel 52 each inside every cell culture chamber, each liquid hose line 27A or 27B or 27C or 27D or 27E respectively discharges into a corresponding liquid channel 51 inside every cell culture chamber, whereas every gas hose line 26A or 26B or 26C or 26D or 26E respectively discharges into a corresponding gas channel 50 of every cell culture chamber.

From the sixth cell culture chamber VI, the liquid outlet lines 27F and 28F and a gas outlet line 26F branch off.

As a result, all cell cultures in the six cell culture chambers I to VI can be continuously supplied with freely selectable, defined, liquid media as well as subjected to a constant, continuous gassing via the gassing system D according to FIG. 1, as already explained in detail above.

Please note that FIG. 2 only shows one of many possible directions of flow for liquids and gases. By means of the flexible hose line systems for liquids and gases explained above, other cell culture chamber combinations than the ones shown in FIG. 2 can be controlled as well.

Another particularly important aspect is that the cell culture chamber group A as a whole is permanently connected to the monitoring system F according to FIG. 1 so that all relevant parameters can be measured in the relevant cell culture chamber 20 by means of accordingly assigned sensors for the entire duration of a test. For this reason, each of the cell culture chambers 20 is fitted inside with a corresponding channel 53 for the connection of sensors. In detail, the first cell culture chamber I is connected, via a line 44A, the second cell culture chamber II, via a line 44B, and the third cell culture chamber III, via a line 44C, to a line 44, while the fourth cell culture chamber IV is connected via a line 43A, the fifth cell culture chamber V, via a line 43B, and the sixth cell culture chamber VI, via a line 43C, to a line 43. The lines 43 and 44 lead to a junction point 42 that is connected, via a line 41, to the monitoring system F according to FIG. 1.

The sensors that are arranged inside each cell culture chamber 20 (the chambers I to VI in this application example) and that are not represented in detail here enable a permanent measuring of the relevant parameters; the relevant measured values are then transmitted by the monitoring system F to the computer-controlled monitoring and control system G according to FIG. 1 for corresponding further processing and a subsequent actuation of the feedback control elements also contained in the monitoring and control system G.

FIG. 2 further shows that the top of every cell culture chamber 20 of the cell culture chamber group A is fitted with a round window 20A with a glass pane; this enables illumination of the entire area of the cell culture contained in the relevant cell culture chamber 20, as it has already been explained in detail by means of FIG. 1 above.

As for the rest, the cell culture chamber as such is the subject matter of a German patent application of the same applicant with the designation “Cell culture chamber for a cell culture system” (official application number . . . ).

The device for cultivating cells according to the invention offers the opportunity to simulate highly complex, biological processes in real time and almost under in-vivo conditions.

A special advantage of the device according to the invention is that it can be used, in particular, to investigate cellular functions, to examine the action of drugs, to develop drugs, to co-cultivate different types of cells and parts of tissue, to carry out organotypic studies, to observe tumor cells in a typical environment or to carry out toxicological studies.

For reasons of completeness, please note that the device according to the invention can be modified in such a way that the above-mentioned feedback control mechanism (control elements, control algorithms) is not started; in practice, this means that the cell cultivation process is left alone without the incubation conditions being controlled via the feedback control mechanism.

For this operating model of the device for cultivating cells according to the invention, the cell culture parameters are set a priori but are not changed during the cell cultivation process although they are measured continuously with the operating model explained last.

1. Device for cultivating cells of the most diverse type, particularly human or animal cells, one culture each of cells of at least one specific type being established in a defined environment and the cells of the relevant culture being supplied with assigned, liquid nutrient media, growth factors, gases and the like in the process, characterized by the fact that the device (30) is equipped with cell cultivation equipment and incubation equipment of such a kind that they enable the cells established in at least one cell culture chamber (20) of the device (30) to adjust the living and growth conditions necessary in the individual case themselves.

2. Device according to claim 1, characterized by the combination of the following features:
a) Equipment (C) for starting a flow of freely selectable, defined, liquid media in the at least one cell culture chamber (20) in order to ensure a continuous supply for the cells that have been established there;
b) Equipment (D) for starting a flow of different gases with freely selectable concentrations into the at least one cell culture chamber (20) in order to ensure a constant, continuous gassing of the cells that have been established there;
c) Equipment (E) for a regulated and/or controlled heating of the at least one cell culture chamber (20) in such a way so as to ensure a constant temperature in there over the duration of an experiment;
d) Equipment (B) for a permanent microscopic observation of the cells established inside the at least one cell culture chamber (20), without samples of the cell culture being taken over the duration of an experiment;
e) Equipment (F) for a permanent measuring of all relevant cell culture parameters by means of suitable sensors integrated in the at least one cell culture chamber (20), and
f) The feedback control elements assigned to at least one cell culture chamber (20) for the purpose of optimizing the incubation conditions in the cell culture chamber (20).

3. Device according to claim 1, characterized by the fact that the device (30) is equipped with a given number of cell culture chambers (20) connected in series.

4. Device according to claim 1, characterized by the fact that the device (30) is equipped with a given number of cell culture chambers (20) that are connected in parallel.

5. Device according to claim 2, characterized by means that serve to vary the type of liquid media and/or their directions of flow and/or their distribution and/or their flow volumes during the duration of a test.

6. Device according to claim 2, characterized by equipment that serves to vary the type of gases and/or their directions of flow and/or distribution and/or gassing concentration over the duration of an experiment.

7. Device according to claim 1, characterized by an equipment (25) that serves to permanently measure the temperature in the at least one cell culture in the at least one cell culture chamber (20) and to enter it as an actual temperature value into a corresponding temperature adjusting and/or control circuit so that the heating of the cell culture chamber (20) is adjusted and/or controlled accordingly.

8. Device according to claim 1, characterized by at least one cell culture chamber (20) in which a gas-permeable membrane has been arranged in such a way that one cell culture of different types each may be established for the purpose of a direct co-cultivation of both cell cultures on both sides of this membrane; equipment for the starting of a first flow of media to the first side of the membrane, that means to the apical side with the first cell culture, and a second flow of media that is different from the first flow of media to the other side of the membrane, that means the basolateral side with the second cell culture, being provided.

9. Device according to claim 2, characterized by a video-supported microscopic observation system (B) for observing the at least one cell culture in the at least one cell culture chamber (20).

10. Device according to claim 2, characterized by a computer-controlled monitoring and control system (G), to which all data obtained by means of
A permanent microscopic observation of the at least one cell culture inside the at least one cell culture chamber (20) and/or
A permanent measuring of the relevant cell culture parameters and/or
A permanent measuring of the temperature in the at least one cell culture inside the at least one cell culture chamber (20)
can be transmitted for further processing and a subsequent corresponding actuation of the feedback control elements.

11. Device according to claim 2, characterized by a software-aided measuring system for a permanent measuring of the relevant cell culture parameters.

12. Device according to claim 1, characterized by the fact that the cell culture chambers (20) are integrated into a closed cell chamber group (A) arranged on a base (21) that constitutes a heating system (E) for incubation.

13. Device according to claim 1, characterized by the use of the device for an indirect co-cultivation, different biological systems (that means types of tissue/cells) being connected in series in suitable cell culture chambers (20).
